(19) United States
(12) Patent Application Publication
(10) Pub. No.: US 2024/0189263 A1
(43) Pub. Date: Jun. 13, 2024

(54) DERIVATIZED C60 MOLECULES AND METHODS AND MATERIALS FOR USING DERIVATIZED C60 MOLECULES TO REDUCE RADIATION INJURY

(71) Applicants: Mayo Foundation for Medical Education and Research, Rochester, MN (US); AGNI Medical LLC, Jacksonville, FL (US)

(72) Inventors: Sunil Krishnan, Ponte Vedra Beach, FL (US); Yuri Mackeyev, Jacksonville, FL (US); Elena Vishnyakova, Pompano Beach, FL (US)

(21) Appl. No.: 18/286,297

(22) PCT Filed: Jun. 24, 2022
(86) PCT No.: PCT/US2022/034917

§ 371 Date: Oct. 10, 2023

Related U.S. Application Data
(60) Provisional application No. 63/215,967, filed on Jun. 28, 2021.

(51) Int. Cl. A61K 31/165; A61P 39/00

(52) U.S. Cl. CPC A61K 31/165; A61P 39/00

(57) ABSTRACT

This document provides derivatized C₆₀ molecules (e.g., C₆₀-serinol) and methods and materials for using derivatized C₆₀ molecules (e.g., C₆₀-serinol) to reduce radiation injury. For example, methods and materials for using derivatized C₆₀ molecules (e.g., C₆₀-serinol) to reduce, prevent, and/or mitigate radiation injury (e.g., radiation induced oxidative stress and/or DNA damage) to cells (e.g., human cells) or to a mammal (e.g., a human) during, for example, outer space missions are provided.

### CROSS-REFERENCE TO RELATED APPLICATIONS

This application claims the benefit of U.S. Patent Application Ser. No. 63/215,967, filed on Jun. 28, 2021. The disclosure of the prior application is considered part of (and is incorporated by reference in) the disclosure of this application.

### STATEMENT REGARDING FEDERAL FUNDING

This invention was made with Government support under 80NSSC20C0529 awarded by NASA. The Government has certain rights in the invention.

### BACKGROUND

### 1. Technical Field

This document relates to derivatized C₆₀ molecules (e.g., C₆₀-serinol, also referred to herein as C₆₀-ser) and methods and materials involved in using derivatized C₆₀ molecules (e.g., C₆₀-serinol) to reduce radiation injury. For example, this document provides methods and materials for using derivatized C₆₀ molecules (e.g., C₆₀-serinol) to reduce, prevent, and/or mitigate radiation injury (e.g., radiation induced oxidative stress) to cells (e.g., human cells) or to a mammal (e.g., a human).

### 2. Background Information

Exposure to cosmic radiation poses health risks to mammals (e.g., humans). Shielding with protective materials can provide a first line of defense, but additional strategies may be required to protect mammals from prolonged radiation exposure.

### SUMMARY

This document provides derivatized C₆₀ molecules (e.g., C₆₀-serinol) and methods and materials for using derivatized C₆₀ molecules (e.g., C₆₀-serinol) to reduce radiation injury. For example, this document provides methods and materials for using derivatized C₆₀ molecules (e.g., C-serinol) to reduce, prevent, and/or mitigate radiation injury (e.g., radiation-induced oxidative stress or DNA damage) to cells (e.g., human cells) or to a mammal (e.g., a human) during, for example, outer space missions. In some cases, this document provides methods and materials for using derivatized C₆₀ molecules (e.g., C₆₀-serinol) to prolong a mammal's survival after being exposed to a high dose of radiation (e.g., greater than 0.5 Gy).

As demonstrated herein, a composition containing C₆₀-serinol can be administered to a mammal (e.g., a human) before, during, and/or after exposure to radiation (e.g., radiation from about 0.5 Gy to about 8 Gy or from about 0.5 Gy to about 6 Gy or from about 0.7 Gy to about 6 Gy) to reduce the severity and/or lethality of the radiation-induced injury that normally results from that exposure.

In general, one aspect of this document features a method for prolonging survival of a mammal exposed to a lethal level of radiation. The method comprises (or consists essentially of or consists of) administering a composition comprising C₆₀-serinol to the mammal, wherein the mammal survives longer than if the mammal was not administered the composition. The mammal can be human. The lethal level of radiation can be greater than 5 Gy. The lethal level of radiation can be from about 5 Gy to about 10 Gy. The composition can be administered via injection. The composition can be administered orally. The composition can be administered within two hours of being exposed to the lethal level of radiation. The composition can be administered from about one minute to about 24 hours after being exposed to the lethal level of radiation.

In another aspect, this document features a method for prolonging survival of a mammal expected to be exposed to a lethal level of radiation. The method comprises (or consists essentially of or consists of) administering a composition comprising C₆₀-serinol to the mammal before the mammal is exposed to the lethal level of radiation, wherein the mammal survives longer than if the mammal was not administered the composition. The mammal can be human. The lethal level of radiation can be greater than 5 Gy. The lethal level of radiation can be from about 5 Gy to about 10 Gy. The composition can be administered via injection. The composition can be administered orally. The composition can be administered at least one hour before the mammal is exposed to the lethal level of radiation. The composition can be administered from about one minute to about 24 hours before the mammal is exposed to the lethal level of radiation.

In another aspect, this document features a method for reducing the severity of radiation-induced injury. The method comprises (or consists essentially of or consists of) administering a composition comprising C₆₀-serinol to a mammal exposed to a damaging level of radiation, wherein the severity of radiation-induced injury of the mammal is less than the severity of radiation-induced injury if the mammal was not administered the composition. The mammal can be human. The damaging level of radiation can be greater than 0.5 Gy. The damaging level of radiation can be from about 0.5 Gy to about 6 Gy. The composition can be administered via injection. The composition can be administered orally. The composition can be administered within two hours of being exposed to the damaging level of radiation. The composition can be administered from about one minute to about 24 hours after being exposed to the damaging level of radiation.

In another aspect, this document features a method for reducing the severity of radiation-induced injury. The method comprises (or consists essentially of or consists of) administering a composition comprising C₆₀-serinol to a mammal prior to exposure to a damaging level of radiation, wherein the mammal is exposed to the damaging level of radiation, and wherein the severity of radiation-induced injury of the mammal is less than the severity of radiation-induced injury if the mammal was not administered the composition. The mammal can be human. The damaging level of radiation can be greater than 0.5 Gy. The damaging level of radiation can be from about 0.5 Gy to about 6 Gy. The composition can be administered via injection. The composition can be administered orally. The composition can be administered two hours before being exposed to the damaging level of radiation. The composition can be administered from about one minute to about 24 hours before being exposed to the damaging level of radiation.

In another aspect, this document features a method for treating a mammal to reduce the number of reactive oxygen species in the mammal following radiation exposure. The method comprises (or consists essentially of or consists of) administering a composition comprising C60-serinol to the mammal before, during, or after exposure to a damaging level of radiation, wherein the mammal is exposed to the damaging level of radiation, and wherein the number of reactive oxygen species in the mammal following the radiation exposure is less than the number of reactive oxygen species in the mammal if the mammal was not administered the composition. The mammal can be human. The damaging level of radiation can be greater than 0.5 Gy. The damaging level of radiation can be from about 0.5 Gy to about 6 Gy. The composition can be administered via injection. The composition can be administered orally. The composition can be administered two hours before being exposed to the damaging level of radiation. The composition can be administered from about one minute to about 24 hours before being exposed to the damaging level of radiation. The composition can be administered during exposure to the damaging level of radiation. The composition can be administered within two hours of being exposed to the damaging level of radiation. The composition can be administered from about one minute to about 24 hours after being exposed to the damaging level of radiation.

In another aspect, this document features a composition comprising (or consisting essentially of or consisting of) C₆₀-serinol. The composition can further comprise an aqueous or non-aqueous sterile injection solution. The composition can comprise at least one item selected from the group consisting of anti-oxidants, buffers, bacteriostats, solutes, suspending agents, and thickening agents.

In another aspect, this document features a method of making C₆₀-serinol. The method comprises (or consists essentially of or consists of) (a) reacting serinol malonate tetraacetate with [60]fullerene in the presence of 1,8-diazabicyclo[5.4.0]undec-7-ene and perbromomethane in toluene to yield C₆₀-serinol acetate; and (b) reacting the C₆₀-serinol acetate with hydrochloric acid in an aqueous solution of dioxane to yield C₆₀-serinol. The reacting serinol malonate tetraacetate with [60]fullerene can comprise reacting serinol malonate tetraacetate and [60]fullerene in a 6:1 to 15:1 molar ratio. The reacting serinol malonate tetraacetate with [60]fullerene can comprise reacting serinol malonate tetraacetate and [60]fullerene in an 8:1 molar ratio. The method can comprise reacting serinol malonate with acetic anhydride to yield serinol malonate tetraacetate. The method can comprise reacting dimethyl malonate and serinol in isopropyl alcohol to yield serinol malonate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows an example chemical structure of [60]fullerene.

FIG. 2 shows an example schematic of the synthesis of C₆₀-serinol.

FIG. 3A shows an example scanning electron microscopy (SEM) image of aggregates of C₆₀-ser.

FIG. 3B shows an enlargement of the SEM image of FIG. 3A.

FIGS. 4A-4D show dynamic light scattering heatmaps of C₆₀-ser created by the combination of multiple normalized particle number weighted distribution plots, in deionized water at a concentration of 0.1 mg/mL (A) and 10 mg/mL (B), and in phosphate buffered saline at a concentration of 0.1 mg/mL (C) and 10 mg/mL (D).

FIGS. 5A-5B shows an analysis of zeta-potentials of C₆₀-ser aggregates at a concentration of 0.1 mg/mL (A) and 10 mg/mL (B) in deionized water.

FIG. 6A shows the percent viability of N2A cells over time and at increasing concentrations of C₆₀-ser.

FIG. 6B shows the percent viability of HUVEC cells over time and at increasing concentrations of C₆₀-ser.

FIG. 6C shows the percent viability of NIH3T3 cells over time and at increasing concentrations of C₆₀-ser.

FIG. 6D shows the percent viability of HepG2 cells over time and at increasing concentrations of C₆₀-ser.

FIG. 7A shows the relative fluorescent intensity of H₂-DCFDA probes in irradiated N2A cells treated with C₆₀-ser, TBHP, NAC, and combinations thereof.

FIG. 7B shows the relative fluorescent intensity of H2-DCFDA probes in irradiated HUVEC cells treated with C₆₀-ser, TBHP, NAC, and combinations thereof.

FIG. 7C shows the relative fluorescent intensity of H2-DCFDA probes in irradiated NIH3T3 cells treated with C₆₀-ser, TBHP, NAC, and combinations thereof.

FIG. 7D shows the relative fluorescent intensity of H2-DCFDA probes in irradiated HepG2 cells treated with C₆₀-ser, TBHP, NAC, and combinations thereof.

FIG. 8A shows example images of irradiated control and C₆₀-ser pretreated N2A cells, with immunofluorescence staining of γH2AX and DAPI images overlaid with each other (i) and γH2AX foci alone (ii).

FIG. 8B shows example images of irradiated control and C₆₀-ser pretreated HUVEC cells, with immunofluorescence staining of γH2AX and DAPI images overlaid with each other (i) and γH2AX foci alone (ii).

FIG. 8C shows example images of irradiated control and C₆₀-ser pretreated NIH3T3 cells, with immunofluorescence staining of γH2AX and DAPI images overlaid with each other (i) and γH2AX foci alone (ii).

FIG. 8D shows example images of irradiated control and C₆₀-ser pretreated HepG2 cells, with immunofluorescence staining of γH2AX and DAPI images overlaid with each other (i) and γH2AX foci alone (ii).

FIG. 9A shows the foci distribution of N2A cells following exposure to 0.5 and 1 Gy of 227 MeV proton radiation with or without 100 μM C₆₀-ser FIG. 9B shows the foci distribution of HUVEC cells following exposure to 0.5 and 1 Gy of 227 MeV proton radiation with or without 100 μM C₆₀-ser.

FIG. 9C shows the foci distribution of NIH3T3 cells following exposure to 0.5 and 1 Gy of 227 MeV proton radiation with or without 100 μM C₆₀-ser.

FIG. 9D shows the foci distribution of HepG2 cells following exposure to 0.5 and 1 Gy of 227 MeV proton radiation with or without 100 μM C₆₀-ser.

FIG. 10A shows the results of the classic clonogenics survival analysis of irradiated N2A cells pre-treated with C₆₀-ser.

FIG. 10B shows the results of the classic clonogenics survival analysis of irradiated NIH3T3 cells pre-treated with C₆₀-ser.

FIG. 10C shows the results of the classic clonogenics survival analysis of irradiated HepG2 cells pre-treated with C₆₀-ser.

FIG. 11A shows the oxygen consumption rates of irradiated N2A cells in a Seahorse mitochondrial respiration experiment.

FIG. 11B shows the oxygen consumption rates of irradiated HUVEC cells in a Seahorse mitochondrial respiration experiment.

FIG. 11C shows the oxygen consumption rates of irradiated NIH3T3 cells in a Seahorse mitochondrial respiration experiment.

FIG. 11D shows the oxygen consumption rates of irradiated HepG2 cells in a Seahorse mitochondrial respiration experiment.

FIG. 12 shows the percent survival rate of mice exposed to whole body radiation, with and without treatment with C₆₀-ser.

FIG. 13 shows the results of a clonogenics assay of irradiated HCT116 colorectal cancer cells, with and without treatment with C₆₀-ser.

FIG. 14 shows the normalized tumor volumes of syngeneic mouse models of pancreatic cancer treated with capecitabine or capecitabine and C₆₀-ser and exposed to radiation.

### DETAILED DESCRIPTION

This document provides derivatized C₆₀ molecules (e.g., C₆₀-serinol) and methods and materials for using derivatized C₆₀ molecules (e.g., C₆₀-serinol) to reduce radiation injury. For example, a composition containing C₆₀-serinol can be administered to a mammal (e.g., a human) before, during, and/or after exposure to radiation (e.g., radiation from about 0.5 Gy to about 8 Gy or from about 0.5 Gy to about 6 Gy or from about 0.7 Gy to about 6 Gy) to reduce the severity and/or lethality of the radiation-induced injury that normally results from that exposure. In some cases, a composition containing C₆₀-serinol can be administered to a mammal before, during, and/or after exposure to radiation (e.g., radiation from about 0.5 Gy to about 8 Gy or from about 0.5 Gy to about 6 Gy or from about 0.7 Gy to about 6 Gy) to reduce the severity of oxidative stress that the mammal experiences and/or to reduce the severity of DNA damage that occurs within the mammal. In some cases, a composition containing C₆₀-serinol can be administered to a mammal before, during, and/or after exposure to radiation (e.g., radiation from about 0.5 Gy to about 8 Gy or from about 0.5 Gy to about 6 Gy or from about 0.7 Gy to about 6 Gy) to reduce the lethality of the radiation exposure for that mammal and/or to prolong the survival time of that mammal.

Radiation exposure, whether acute or prolonged, can result in negative health effects across multiple organs and systems. For example, exposure to radiation can result in accelerated aging, an increased risk of cancer, late and early central nervous system effects, cardiovascular disease, and damage to hematopoietic, gastrointestinal, cardiovascular, and integumentary systems. These systems and the organs therein interact with each other under physiological conditions to maintain homeostasis. Disruption of this homeostasis can precipitate progressive physiological failure of these interdependent organ systems. Identifying a common pathway that is dysregulated by radiation exposure and targeting this specific pathway offers an opportunity to protect or treat multiple organ systems and disease processes simultaneously. In turn, the resultant restoration of physiological homeostasis across organ types can improve overall health and well-being. To date, there is no compound or treatment that has been shown to work across disease sites and organ systems to protect these organs and systems from radiation-induced injury. Rather, individual agents have been shown to mitigate radiation-induced injury in specific tissues, for example, granulocyte colony stimulating factor and granulocyte-macrophage colony stimulating factor have been shown to mitigate hematopoietic syndrome, but no turnkey class solution exists for the adverse molecular and physiological effects of radiation. Accordingly, a countermeasure agent that targets a common process across multiple disease states is desirable. One such target process is oxidative stress induced by free radical generation within cells and tissues exposed to radiation, which can result in an increase in DNA damage and cellular senescence or accelerated aging.

Oxidative stress can overwhelm cellular homeostasis and repair mechanism. Cellular mitochondria produce endogenous reactive oxygen species in normal cells, however, this oxidative stress and its downstream damage (especially DNA damage) is readily detected and repaired by cells via homeostatic mechanisms. For instance, sublethal DNA damage is faithfully repaired by a host of repair processes. If repair machinery is overwhelmed or repair is defective, cells trigger apoptotic death pathways to commit suicide. This decision point between active repair and programmed self-destruction is a part of homeostasis across organ systems responding to genotoxic stress. Radiation injury is one such form of genotoxic stress that arises from ionization of atoms along a radiation path. The radiation can create oxygen free radicals, which trigger DNA damage and cell death. Since oxidative stress, beyond that generated endogenously by mitochondria, is a primary component of radiation injury, the oxidative stress has ramifications across many cell types and physiologies.

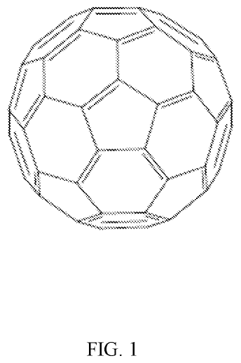

As described herein, compositions containing a [60]fullerene derivative (e.g., C₆₀-serinol) can be used as highly potent and effective antioxidants in either or both pre-exposure and post-exposure settings to scavenge free radicals and reduce acute and chronic damage from exogenous oxidative stress. For example, compositions containing C₆₀-serinol can be administered before, during, and/or after radiation exposure to reduce radiation-induced injury to a mammal exposed to damaging and/or lethal radiation. [60]fullerene, also known as C₆₀ or buckminsterfullerene, resembles a soccer ball with a closed mesh topology. The molecule includes pentagonal and hexagonal rings of carbon in which no two pentagons share an edge. The molecule is highly symmetrical, and has an unusual aromatic structure that includes the delocalization of 48 π electrons over the mesh structure. FIG. 1 shows an example chemical structure of [60]fullerene. A [60]fullerene molecule is about 1 nm in diameter, so it can be considered to bridge the gap between molecules and nanoparticles. [60]fullerene is a potent antioxidant, however, it is not readily formulated for biological applications due to its extreme lipophilicity and lack of solubility in aqueous media. However, as described herein, the properties of [60]fullerene can be altered by derivatization. For example, derivatizing [60]fullerene with serinol results in a molecule that is amphiphilic and non-toxic in nature. In contrast, other derivatized [60]fullerenes can exhibit dose- and time-dependent toxicity.

The serinol-derived [60]fullerene (C₆₀-ser) molecule described herein is electrostatically neutral and hydrophilic. C₆₀-ser has a hydrophobic core and a hydrophilic surface and can be considered an amphiphilic molecule. For example, the hexakis-C₆₀-ser adduct (with six serinol derived functional groups) has 24 hydroxyl groups on its surface, which shield the electron-rich aromatic molecular center. Further, the hydroxyl groups maintain a number of relatively stable layers of closely bound water molecules, i.e., a hydration sphere. Accordingly, there is a continuous network of hydrogen bonds surrounding the C₆₀-ser. Further, C₆₀-ser has a high solubility in water, greater than 250 mg/mL.

In some cases, C₆₀-ser molecules can be synthesized via a [2+1]cycloaddition (cyclopropanation) reaction where an in situ produced bromomalonate derivative links to the fullerene at the junction of two hexagons in the presence of a base to relieve steric strain, also known as a Bingel reaction. This electrophilic addition at a 6,6-double bond converts sp²-hybridized carbons to sp³-hybridized carbons and reduces the angular strain. The resultant molecule is more stable since the bond angles are slightly smaller in the sp³ orbitals and need to bend less.

In some cases, the C₆₀-ser molecules can by synthesized via a reaction with serinol malonate tetraacetate. The ratio of serinol malonate tetraacetate can be between 6:1 and 15:1, for example 8:1. Higher ratios of serinol malonate tetraacetate can result in an increased yield of the hexakis-adduct. Subsequent acetate protection group removal can create the highly water soluble fullerene, C₆₀-ser. These synthetic approaches can result in C₆₀-ser.

The C₆₀-ser molecule has a number of advantages, including unique physical properties, for example, stability and solubility in aqueous solutions. As described herein, the presence of hydroxyl groups on the surface of the C₆₀-ser molecule increases its aqueous solubility. The solubility of C₆₀-ser (>250 mg/mL) is greater than other known fullerene derivatives.

In addition, C60-ser has distinct biological behavior due in part to its unique solubility and physical properties. The soluble C₆₀-ser molecules can spontaneously form labile aggregates when dissolved in water. The C₆₀-ser molecules exist in a dynamic equilibrium state, seamlessly flipping between the conjugate monomers (˜3 nm) and aggregates (100-2000 nm). The aggregate formation and equilibrium process can be influenced by hydrogen bonding, π-π stacking interactions, and hydrophobic forces. For example, water molecules surrounding the carbon shell reorient and replace each other in a way that maintains the maximum number of hydrogen bonds. In addition, hydrophobic forces can repel water from the non-polar regions of the C₆₀-ser molecule. These forces can contribute to the dynamic equilibrium described herein. Without being bound by any particular theory, it is believed that the dynamic equilibrium states enable the conjugates to use passive diffusion and active transport (e.g., endocytosis, transcytosis) for transport into and through cells. This can allow efficient and seamless shuttling of the molecule from the vasculature to tissues including the brain. In addition, the C₆₀-ser molecules have excellent pharmacokinetics, due to long circulation time as a result of evasion of opsonization and reticuloendothelial capture, as well as sub-5 nm size-dependent renal elimination. Further, the robust radiation mitigation effects in vivo of C₆₀-ser demonstrate that intravenously administered C₆₀-ser can protect animals from whole-body radiation. Moreover, C₆₀-ser does not protect tumor cells from radiation induced injury. Tumor cells in vitro and in vivo remain susceptible to radiation induced injury, even when pretreated with C₆₀-ser.

The C₆₀-ser molecules are non-toxic, and cell lines treated with C₆₀-ser maintain their viability, even at high doses of C₆₀-ser. Further, C₆₀-ser can mitigate proton radiation damage to cells, as described herein. For example, the C₆₀-ser molecules were evaluated for their ability to mitigate or prevent radiation injury to normal epithelial, endothelial, and mesenchymal cells. A number of radiation energies, qualities, and doses were investigated in multiple cell lines, in combination with C₆₀-ser, to evaluate C₆₀-ser mediated radiation protection and/or mitigation. Several representative cell lines were used, including HepG2 cells (epithelial liver), Neuro-2a (N2A, neuroblastoma cells representative of normal neurons), NIH3T3 cells (fibroblasts), and HUVEC cells (endothelial cells) as representative normal cells across a range of tissue types and organ systems that are often affected by radiation injury. Different concentrations of C₆₀-ser were administered for different time periods before or after low dose radiation with protons (e.g., from about 0.5 Gy to about 1 Gy). Cell lines pretreated with C₆₀-ser exhibit reduced DNA damage in response to ionization, as well as an increase in survivability compared to control cells, as measured by a clonogenics assay.

In addition, the molecular mechanisms of C₆₀-ser mediated reduction of oxidative stress and energy metabolism were investigated. Representative cell types were used to evaluate overall oxidative stress levels with radiation in the presence or absence of C₆₀-ser. The overall oxidative stress levels were evaluated using an H₂-DCFDA reagent, and cells pre-treated with C₆₀-ser exhibited less ROS compared to controls. Further, energy metabolism in representative cells lines was evaluated using an Agilent Seahorse platform for real-time measurement of oxygen consumption rate and extracellular acidification rate. Cells treated with C₆₀-ser and radiation show a reduced mitochondrial respiratory capacity relative to control, indicating less damage to the C₆₀-ser treated cells.

As described herein, C₆₀-ser can be used to treat radiation exposure. For example, C₆₀-ser can be used as a prophylactic treatment before known radiation exposure risks such as space or air travel. Astronauts as well as airline workers can be exposed to cosmic radiation and/or solar flares, and pre-treatment with C₆₀-ser can help prevent or mitigate radiation-induced injury. This can be especially beneficial in prolonged exposure settings, for example, astronauts on extended missions or journeys, such as a journey to Mars. Further, C₆₀-ser can be administered to emergency workers or other personnel that may be exposed to radiation in their working environment.

In some cases, C₆₀-ser can be administered after unexpected exposure to radiation, for example, in response to a failure of a primary physical shield, a solar flare, nuclear accidents, and/or a radiation-emitting weapon. As described herein, administration of C₆₀-ser protected mice from whole body x-ray radiation-induced death when given systemically 12 hours after radiation. Without being bound by any particular theory, it is believed that this protection is the result of extremely potent free radical scavenging.

The methods and materials provided herein can be used to reduce the severity and/or lethality of a radiation-induced injury caused by any type of radiation exposure. For example, the methods and materials provided herein (e.g., compositions containing C₆₀-serinol) can be used to reduce the severity and/or lethality of a radiation-induced injury caused by cosmic radiation during outer space travel and/or caused by medical procedures (e.g., radio imaging or radiotherapy), exposure to a nuclear reactor, exposure to nuclear waste, proximity to a nuclear explosion, or radon exposure.

The methods and materials provided herein can be used to reduce the severity and/or lethality of a radiation-induced injury caused a level of radiation exposure that is from about 0.5 Gy to about 10 Gy (e.g., from about 0.5 Gy to about 9 Gy, from about 0.5 Gy to about 8 Gy, from about 0.5 Gy to about 7 Gy, from about 0.5 Gy to about 6 Gy, from about 0.5 Gy to about 5 Gy, from about 0.5 Gy to about 4 Gy, from about 0.5 Gy to about 3 Gy, from about 0.5 Gy to about 2 Gy, from about 0.7 Gy to about 8 Gy, from about 0.7 Gy to about 7 Gy, from about 0.7 Gy to about 6 Gy, from about 0.7 Gy to about 5 Gy, from about 0.7 Gy to about 4 Gy, from about 1 Gy to about 10 Gy, from about 1 Gy to about 8 Gy, from about 1 Gy to about 7 Gy, from about 1 Gy to about 6 Gy, from about 2 Gy to about 10 Gy, from about 2 Gy to about 8 Gy, from about 2 Gy to about 7 Gy, from about 2 Gy to about 6 Gy, from about 3 Gy to about 10 Gy, from about 3 Gy to about 8 Gy, from about 3 Gy to about 7 Gy, from about 3 Gy to about 6 Gy, from about 5 Gy to about 10 Gy, from about 6 Gy to about 10 Gy, from about 7 Gy to about 10 Gy, from about 8 Gy to about 10 Gy, from about 9 Gy to about 10 Gy, from about 1 Gy to about 9 Gy, from about 2 Gy to about 8 Gy, from about 3 Gy to about 7 Gy, from about 4 Gy to about 6 Gy, from about 1 Gy to about 3 Gy, from about 3 Gy to about 5 Gy, from about 5 Gy to about 7 Gy, or from about 4 Gy to about 6 Gy).

As described herein, a composition containing C₆₀-ser can be used to reduce radiation-induced injury, for example, oxidative stress and/or DNA damage within a mammal. In some cases, a composition containing C₆₀-ser can be administered to a mammal (e.g., a human) pre- or post-exposure to radiation. A composition containing C₆₀-ser can be administered to a mammal exposed to radiation to reduce the severity of radiation-induced injury (e.g., oxidative stress and/or DNA damaga), to reduce one or more symptoms of the radiation-induced injury, and/or to reduce the number of reactive oxidative species (ROS) present within the mammal. For example, a composition containing C₆₀-ser can be administered to a mammal pre- or post-exposure to radiation to reduce one or more symptoms of the radiation-induced injury by, for example, 10, 20, 30, 40, 50, 60, 70, 80, 90, 95, or more percent. For example, a composition containing C₆₀-ser can be administered to a mammal pre- or post-exposure to radiation to reduce the number of ROS present within the mammal by, for example, 10, 20, 30, 40, 50, 60, 70, 80, 90, 95, or more percent.

Any appropriate mammal exposed to radiation or at risk of being exposed to radiation can be treated as described herein. For example, humans and other primates such as monkeys can be treated with a composition containing C₆₀-ser, before, during, and/or after exposure to radiation. In some cases, dogs, cats, horses, cows, pigs, sheep, mice, and rats can be treated with a composition containing C₆₀-ser before, during, and/or after exposure to radiation as described herein.

As described herein, exposure to radiation can come from a number of sources. For example, astronauts are exposed to cosmic radiation and/or solar flares during space missions. In another example, bioterrorism or weapons that emit radiation can result in radiation exposure, possibly resulting in acute radiation syndromes (ARS). Physical shielding with protective materials can provide a first layer of protection, but additional strategies may be required, especially for prolonged exposure to radiation.

In some cases, C₆₀-ser can be formulated into a composition (e.g., pharmaceutically acceptable composition) for administration to a mammal before, during, and/or after exposure to radiation. For example, a therapeutically effective amount of C₆₀-ser can be formulated together with one or more pharmaceutically acceptable carriers (additives) and/or diluents. A pharmaceutical composition can be formulated for administration in any appropriate dosage form. Examples of dosage forms include solid or liquid forms including, without limitation, gums, capsules, tablets (e.g., chewable tablets, and enteric coated tablets), suppository, liquid, enemas, suspensions, solutions (e.g., sterile solutions), sustained-release formulations, delayed-release formulations, pills, powders, gels, creams, ointments, and granules. Pharmaceutically acceptable carriers, fillers, and vehicles that may be used in a pharmaceutical composition described herein include, without limitation, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing C₆₀-ser can be designed for oral or parenteral (including subcutaneous, intramuscular, intravenous, topical, and intradermal) administration. When being administered orally, a pharmaceutical composition containing C₆₀-ser can be in the form of a pill, syrup, gel, liquid, flavored drink, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing C₆₀-ser can be administered locally or systemically. For example, a composition containing C₆₀-ser can be administered systemically by an oral administration or by injection to a mammal (e.g., a human).

In some cases, a composition containing C₆₀-ser can be administered to deliver an effect amount C₆₀-ser to the mammal (e.g., a human) at an effective frequency and for an effective duration.

An effective amount of a composition containing C₆₀-ser can be any amount that can treat or mitigate the effects of radiation exposure, e.g., oxidative stress and/or DNA damage and/or death, without producing significant toxicity to the mammal. An effective amount of C₆₀-ser can be any appropriate amount. In some cases, an effective amount of C₆₀-ser can be from about 70 mg/kg body weight of a mammal to about 700 mg/kg body weight (e.g., from about 70 mg/kg to about 600 mg/kg, from about 70 mg/kg to about 500 mg/kg, from about 70 mg/kg to about 400 mg/kg, from about 70 mg/kg to about 300 mg/kg, from about 70 mg/kg to about 200 mg/kg, from about 70 mg/kg to about 100 mg/kg, from about 100 mg/kg to about 700 mg/kg, from about 200 mg/kg to about 700 mg/kg, from about 300 mg/kg to about 700 mg/kg, from about 400 mg/kg to about 700 mg/kg, from about 500 mg/kg to about 700 mg/kg, from about 600 mg/kg to about 700 mg/kg, from about 100 mg/kg to about 600 mg/kg, from about 200 mg/kg to about 500 mg/kg, from about 300 mg/kg to about 400 mg/kg, from about 100 mg/kg to about 300 mg/kg, from about 200 mg/kg to about 400 mg/kg, from about 300 mg/kg to about 500 mg/kg, or from about 400 mg/kg to about 600 mg/kg body weight) of a mammal. In some cases, an effective amount of C₆₀-ser can from about 10 nM to about 1000 μM (e.g., about 10 nM to about 900 μM, about 10 nM to about 800 M, about 10 nM to about 700 μM, about 10 nM to about 600 μM, about 10 nM to about 500 M, about 10 nM to about 400 μM, about 10 nM to about 300 μM, about 10 nM to about 200 μM, about 10 nM to about 100 μM, about 10 nM to about 0.5 μM, about 500 nM to about 1000 μM, about 1000 nM to about 1000 μM, about 100 μM to about 1000 μM, about 200 μM to about 1000 μM, about 300 μM to about 1000 μM, about 400 μM to about 1000 M, about 500 μM to about 1000 μM, about 600 μM to about 1000 μM, about 700 μM to about 1000 μM, about 800 μM to about 1000 μM, about 900 μM to about 1000βM, about 100 μM to about 900 μM, about 200 μM to about 800 μM, about 300 μM to about 700 μM, about 400 μM to about 600 μM, about 100 μM to about 300 μM, about 300 μM to about 500 μM, about 500 μM to about 700 μM, or about 700 μM to about 900 μM) plasma concentration. Various factors can influence the actual effective amount used for a particular application. For example, the frequency of administration, duration of treatment, use of multiple treatment agents, route of administration, and severity of the radiation exposure may require an increase or decrease in the actual effective amount administered.

The frequency of administration of a composition containing C₆₀-ser can be any frequency that can treat or mitigate the effects of radiation exposure, e.g., oxidative stress and/or DNA damage and/or death, without producing significant toxicity to the mammal. The frequency of administration can remain constant or can be variable during the duration of treatment. As with the effective amount, various factors can influence the actual frequency of administration used for a particular application. For example, the effective amount, duration of treatment, use of multiple treatment agents, route of administration, and severity of the radiation exposure may require an increase or decrease in administration frequency.

An effective duration for administering a composition containing C₆₀-ser can be any duration that can treat or mitigate the effects of radiation exposure, e.g., oxidative stress and/or DNA damage and/or death, without producing significant toxicity to the mammal. For example, the effective duration can vary from several hours to several days or several days to several weeks, months, or years. Multiple factors can influence the actual effective duration used for a particular treatment. For example, an effective duration can vary with the frequency of administration, effective amount, use of multiple treatment agents, route of administration, severity of the radiation exposure and duration of the radiation exposure.

The invention will be further described in the following examples, which do not limit the scope of the invention described in the claims.

### EXAMPLES

### Example 1—Synthesis of C60-Ser

### Methods

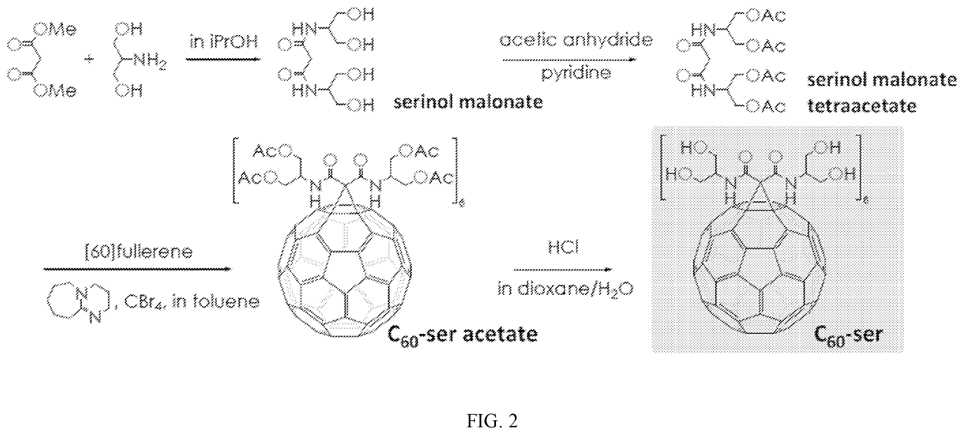

FIG. 2 shows an example schematic of the synthesis of C₆₀-serinol. As shown in FIG. 2, serinol malonate (N,N′-bis-(2-hydroxy-1-hydroxymethyl-ethyl-malonamide) was prepared by reaction of dimethyl malonate and serinol in isopropyl alcohol. The serinol malonate was then converted to an acetyl-protected form, serinol malonate tetraacetate (N,N′-bis-(2-acetoxy-1-acetoxymethyl-ethyl)-malonamide), by reaction with acetic anhydride in pyridine. The serinol malonate tetraacetate was further reacted with [60]fullerene in presence of 1,8-diazabicyclo[5.4.0]undec-7-ene (DBU) and perbromomethane in toluene, in a process called [2+1]cycloaddition. An 8:1 ratio of serinol malonate to [60]fullerene was used. The products of this reaction, hexakis- and pentakis-adducts of C₆₀-ser acetate, were purified by liquid chromatography and converted into the water soluble C₆₀-ser by reaction with hydrochloric acid (HCl) in an aqueous dioxane. The resulting C₆₀-ser was additionally purified by multi-step dialysis using ultra-pure cellulose ester (CE) membranes of 1 and 3.5 kDa pore sizes, the molecular weight cut off values been selected based on an effective size of hydrated C₆₀-ser molecules. The yield of C₆₀-ser was 5-6%.

### Results of Synthesis of C₆₀-Ser

The purity of the resulting C₆₀-ser was analyzed with an Agilent 1200 HPLC equipped with a Kinetex C18 column, 2.6 μm particle size, 100 Å pore size, 100 mm length with 2.1 mm internal diameter (Phenomenex). The C₆₀-ser was also analyzed by an evaporative light scattering detector (ELSD, G4260B) by comparing peak areas of the hexakis C₆₀-ser in a sample. In all of the prepared samples, the combined content of C₆₀-ser products (hexakis- and pentakis-adducts) was found to be greater than 99.8%, with hexakis-adduct of C₆₀-ser content greater than 95.0%.

### Example 2—Stability of C₆₀-Ser

### Methods

Stability of the C₆₀-ser product as prepared in Example 1 was analyzed by measuring the hexakis-adduct peak with high performance liquid chromatography (HPLC) equipped with evaporative light scattering detector (ELSD). The C₆₀-ser was stored as a solid form at 4° C. under nitrogen, as a solution in DI water at 4° C., and as a solution in DI water at room temperature. The stability of the C₆₀-ser in each set of storage conditions was measured at 1, 7, 28, and 120 days (Table 1).

### Results of Stability of C₆₀-Ser

As shown in Table 1, as a solid at 4° C. under nitrogen, no degradation of the hexakis-adduct peak was observed.

TABLE 1
Stability of C₆₀-ser

| | Days | | | |
|---|---|---|---|---|
| Storage Conditions | 1 | 7 | 28 | 120 |
| Solid form at 4° C. | 100 ± 2% | 100 ± 1% | 100 ± 1% | 100 ± 2% |
| Solution at 4° C. | 100 ± 2% | 100 ± 2% | 100 ± 3% | 98 ± 3% |
| Solution at room | 100 ± 2% | 100 ± 2% | 96 ± 3% | 95 ± 4% |
| temperature | | | | |

### Example 3—SEM Analysis of C₆₀-Ser Aggregates

### Methods

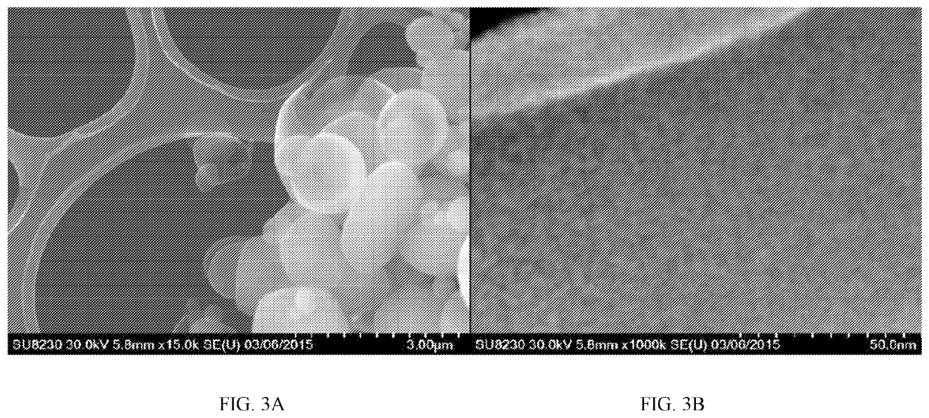

Solutions of C₆₀-ser were frozen with liquid nitrogen and dried under high vacuum while still frozen. FIGS. 3A and 3B show example scanning electron microscopy (SEM) images of aggregates of C₆₀-ser. The aggregates formed when solid C₆₀-ser was dissolved in water or an aqueous salt solution. The aggregates formed within a period of seconds to several hours, after which the system remained in equilibrium.

### Results of SEM Analysis of C₆₀-Ser Aggregates

As shown in FIG. 3A, the aggregates were hollow hulls with internal diameters sized 40-1000 nm. The hollow hulls were formed from a single layer of C₆₀-ser, and were unexpectedly stable. For example, these structures transferred on to an SEM copper grid for microscopic imaging, as seen in FIGS. 3A and 3B. Without wishing to be bound by any particular theory, it is believed that the C₆₀-ser solid preserved its fragile shell-like structures due in part to noncovalent supramolecular hydrogen bonding, dipole-dipole interactions, and/or aromatic π-π stacking interactions. FIG. 3A shows globular C₆₀-ser structures that were hollow spheres or hulls. Some of the spheres were broken, i.e., the hollow center was not fully enclosed. FIG. 3B shows an enlargement of the area enclosed by the rectangle in FIG. 3A.

### Example 4—Dynamic Light Scattering Analysis of C₆₀-Ser Aggregates

### Methods

Dynamic light scattering (DLS) was used to observe the aggregation process of C₆₀-ser. Dynamic light scattering heatmaps were created by combining multiple normalized particle number weighted distribution (PNWD) plots. The PNWD plots compared the log of the relative scattering intensity versus particle size, at angles of 173°. The refractive index was assumed to be n=1.34, the absorption constant to be k=0.001, and the solution viscosity to be that of water, corrected for temperature (η=0.888 mPa s). Plots were collected with a 0.2° C. interval. Each temperature interval was held constant for more than 45 minutes before performing the next measurement, to ensure the presence of an aggregation equilibrium.

### Results of Dynamic Light Scattering Analysis of C₆₀-Ser Aggregates

Initially, when C₆₀-ser was dissolved in water, only individual Cao-ser molecules with diameters of 3-4 nm were observed. Over time, aggregates with diameters in the tens of nms were observed, followed by large aggregates of hundreds of nm after about one minute. Filtering a solution with large aggregates using a 20 nm syringe filter (Whatman 6809-2002, Anotop, 0.02 μm) directly into a DLS cuvette resulted in a solution that was initially free of large aggregates but exhibited a reappearance of aggregates a short time later.

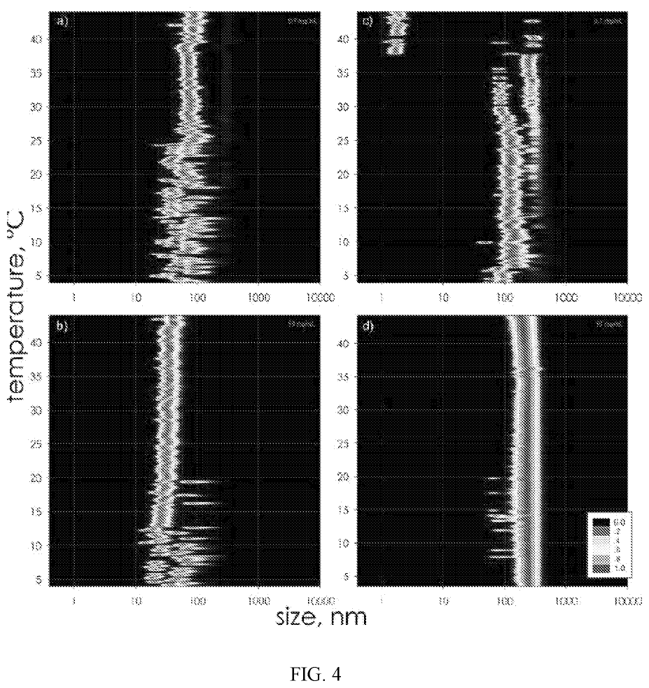

FIGS. 4A-4D illustrate how solution concentration and the temperature affected the aggregate sizes of C₆₀-ser. FIGS. 4A-4D show normalized PNWD DLS plots of C₆₀-ser in deionized water at a concentration of 0.1 mg/mL (FIG. 4A), in deionized water at a concentration of 10 mg/mL (FIG. 4B), in phosphate buffered saline (PBS) at a concentration of 0.1 mg/mL (FIG. 4C), and in PBS at a concentration of 10 mg/mL (FIG. 4D). As shown in these figures, larger-sized aggregates existed at lower temperatures, and low temperatures favored greater polydispersity of aggregate sizes. Table 2 shows the predominant aggregate sizes of C₆₀-ser particles in deionized (DI) water and in PBS.

TABLE 2
Predominate aggregate sizes of C₆₀-ser.

| C₆₀-ser concentration, | Temperature | Predominant aggregate sizes, nm | |
|---|---|---|---|
| mg/mL | (° C.) | in DI water | in PBS |
| 10 | 37 | 193 ± 67 | 233 ± 56 |
| | | 49 ± 13 | |
| | 4 | 245 ± 85 | 290 ± 73 |
| | | 61 ± 21 | |
| | | 22 ± 5.1 | |
| 0.1 | 37 | 312 ± 44 | 260 ± 38 |
| | | 74 ± 11.5 | |
| | 4 | 411 ± 198 | 362 ± 106 |
| | | 108 ± 25 | 102 ± 20 |
| | | 61 ± 11.6 | 65 ± 10.1 |

### Example 5—Zeta-Potential of C₆₀-Ser Aggregates

Classical micelles, for example, micelles of dipolar molecules in solution, normally hold several layers of bond molecules such as a “Stem layer” or “diffusive ion layer.” There is an electrostatic potential at the boundary, called a zeta (ζ)-potential. Although the aggregates formed by C₆₀-ser were not micelles of dipolar molecules, the zeta-potential of the aggregates can be measured.

### Methods

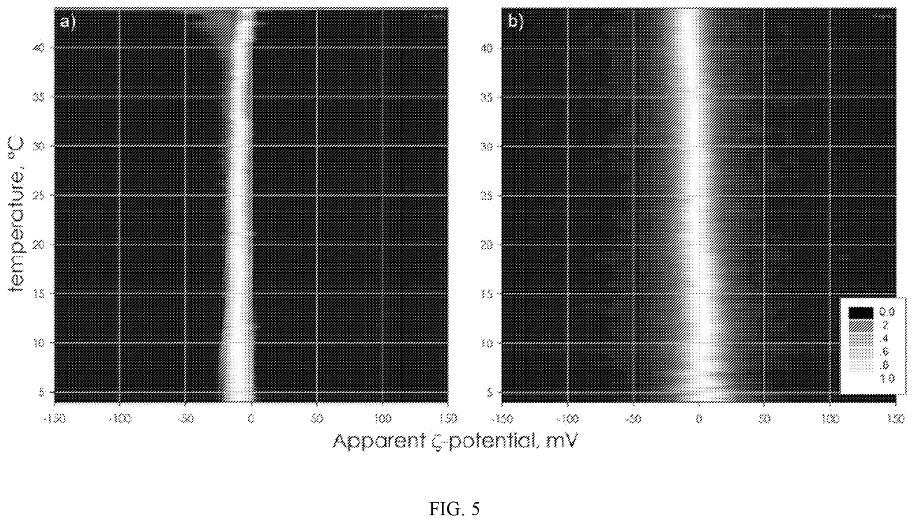

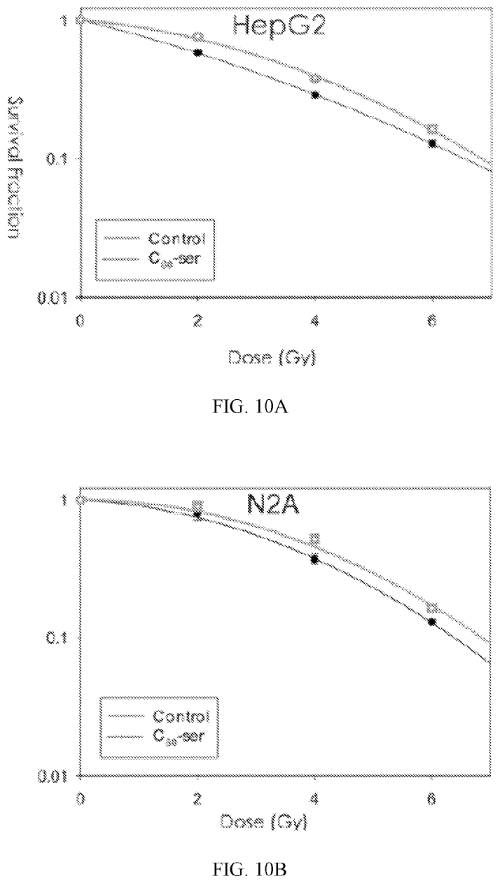

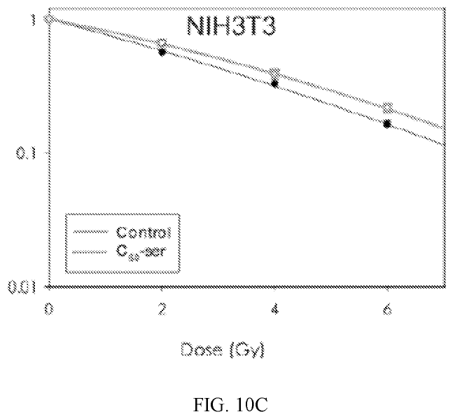

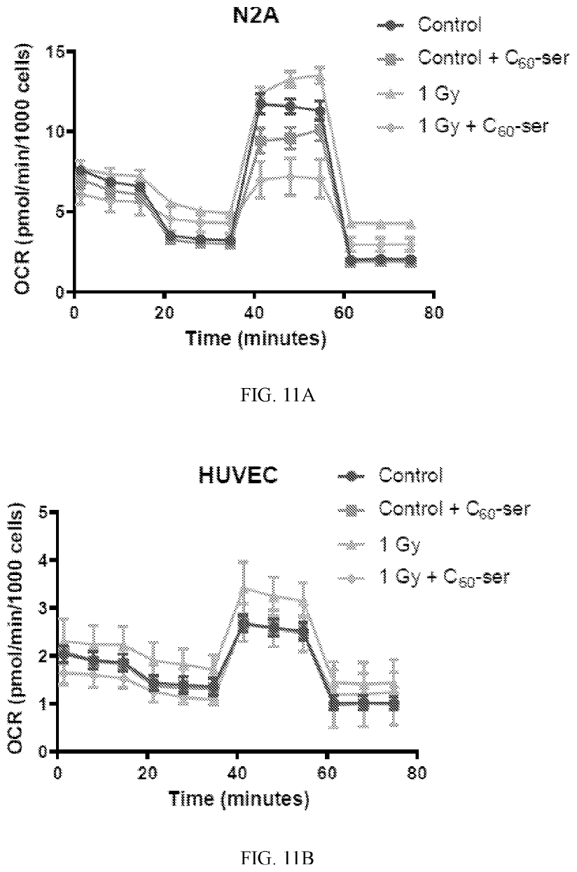

Analysis was performed using a ZEN 3600 Malvern Zetasizer (Worcestershire, UK) equipped with a 633 nm laser. The electrophoretic mobilities (p) of the aggregates was measured by applying an electric field to the C₆₀-ser solution and measuring the speed and direction of the aggregate's motion. Zeta-potentials were calculated using the solvent viscosity (n) and the water dielectric constant (e). To perform the measurements, C₆₀-ser molecules were dissolved in deionized water, and the solution electrical conductivity was adjusted to 25-50 μS/cm by addition of small amounts of neutral 2-(N-morpholino)ethanesulfonic acid (MES) buffer. The zeta-potential of C₆₀-ser aggregates was measured at a concentration of 0.1 mg/mL in deionized water (FIG. 5A) and 10 mg/mL in deionized water (FIG. 5B).

### Results of Zeta-Potential of C₆₀-Ser Aggagates

The zeta-potentials of the C₆₀-ser aggregates were found to be close to zero, as shown in FIGS. 5A-5B. At higher temperatures, for example, greater than 37° C., smaller aggregates became more abundant, and the zeta-potential values turned slightly negative (−7.3 f 12.1 mV). However, this effect was only visible for relatively concentrated solutions (10 mg/mL).

### Example 6—Cytotoxicity of C₆₀-Ser

### Methods

Cytotoxicity of C₆₀-ser was determined by an MTS (3-(4,5-dimethylthiazol-2-yl)-5-(3-carboxymethoxyphenyl)-2-(4-sulfophenyl)-2H-tetrazolim) colorimetric assay on four different cell lines, each representing a different cell type. Specifically, HepG2 (liver epithelium), HUVEC (endothelial cells), N2A (neuroblastoma cells representative of normal neurons), and NIH3T3 (fibroblasts) were analyzed. The cells were seed in a 96-well tissue culture polystyrene plate at different seeding concentrations based on the cell's doubling time. HUVEC cells were seeded at 10,000 cells/well (24 hour experiments), 8,000 cells/well (48 hour experiments), and 5,000 cells/well (72 hour experiments), and incubated for 24 hours in a humidified atmosphere with 5% C02 at 37° C. Six different concentrations of C₆₀-ser (1, 10, 20, 50, 100, and 1000 μM) were administered to each of the cell lines, and incubated for a further 24, 48, or 72 hours to account for the doubling time of each of the cell lines used. Four hours before the termination point (i.e., 20, 44, or 68 hours after administration of C₆₀-ser), the cells were incubated with 20 μL of MTS solution for 4 hours. After incubation with MTS, the optical density (OD) of the resulting formazan solution was read at 490 nm using a microplate reader (Biotek Cytation 5 cell Imaging Multi-Mode reader, Winooski, VT). The cell viability was obtained by normalizing the absorbance of the sample with that of the control wells containing untreated cells. The cell viability was expressed as a percentage, assigning the viability of non-treated cells as 100%. Experiments were performed in pentaplicate.

### Results of Cytotoxicity of C₆₀-Ser

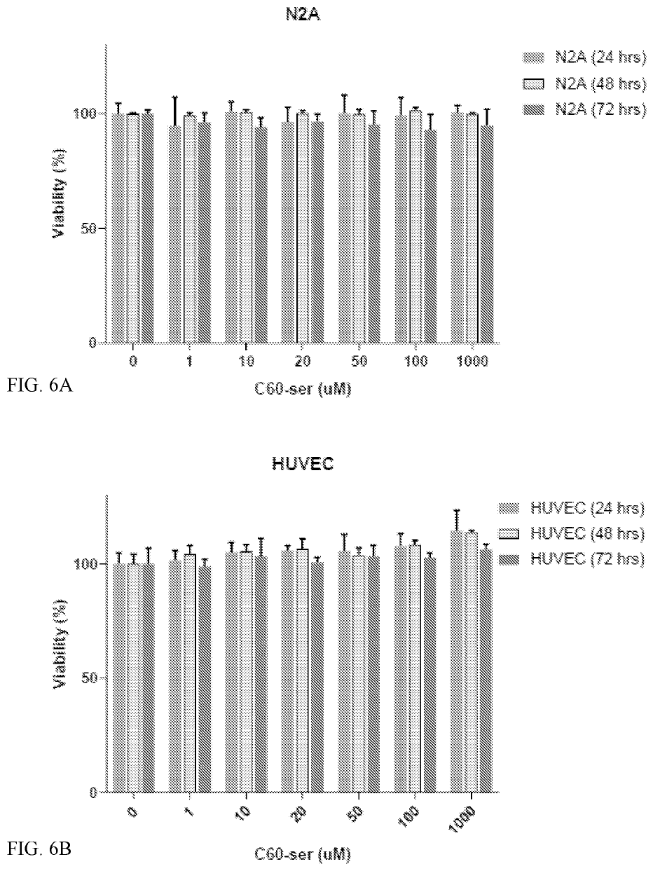

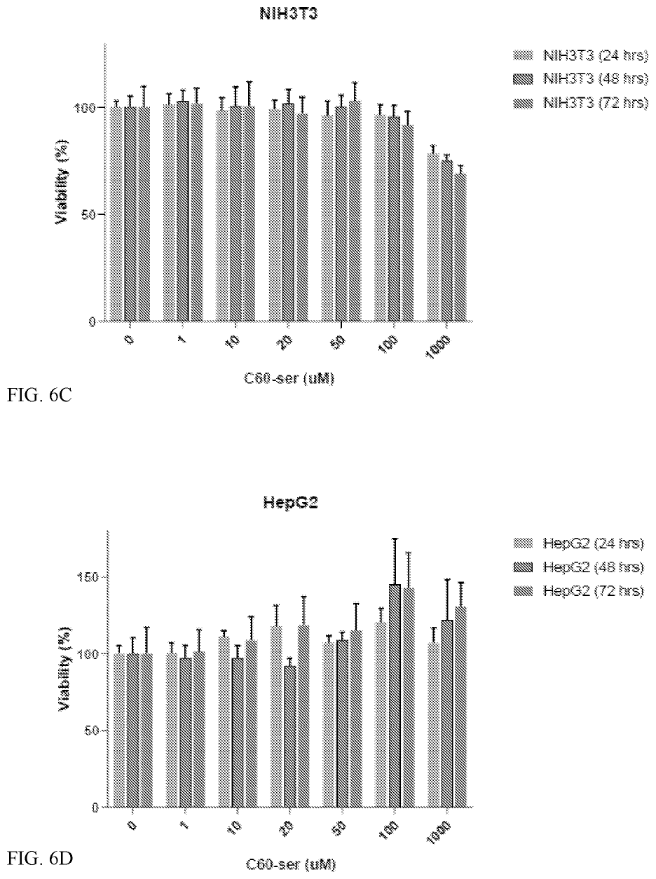

FIGS. 6A-D show the percent viability of different cell lines at different time points and concentrations of C₆₀-ser. FIG. 6A shows the results of N2A cells. FIG. 6B shows the results of HUVEC cells. FIG. 6C shows the results of NIH3T3 cells. FIG. 6D shows the results of HEPG2 cells. For each cell line, treatment with C₆₀-ser did not result in a significant decrease in viability, albeit with a downward trend in NIH3T3 cells at the highest dose studied, 1000 μM.

### Example 7—Evaluation of C₆₀-Ser in Biological Systems Under Proton Irradiation

### Methods

Cell lines were irradiated using pencil beam scanning (PBS) proton beams produced by IBA ProteusOne (Ion Beam Applications, Louvain-la-Neuve, Belgium). Single layer mono-energy 227 MeV pencil beams with uniform spacing of 0.25 cm between each spot were used to create a large field size with uniform dose distribution (±2%) that encompassed all sample wells containing the cells. A fixed source to surface distance (SSD) setup and gantry at 180 degrees was used to delivery 0.5, 1, 2, 4, and 6 Gray (Gy) dose of ionizing radiation to cells. The cells were place at an 8 cm depth, with a linear energy transfer (LET) of approximately 1.7 keV/μm, using solid water slabs as a buildup. The dose per monitor unit (MU) was calibrated in the same configuration as the cell irradiation using an Accredited Dosimetry Calibration Laboratory (ADCL) calibrated parallel plate ion chamber. The monitor units needed to deliver the intended dose were calculated using the measure dose/MU values.

Post radiation, 2′,7′-dichlorodihydrofluorescein diacetate (H₂-DCFDA) probes were used to measure the level of reactive oxygen species (ROS) post-proton radiation (1 Gy, 230 MeV proton beam). The H₂-DCFDA was a reduced form of fluorescein that is cell permeable and not fluorescent on its own. Once the acetate groups of H₂-DCFDA are cleaved by intracellular esterases and oxidized by a reactive oxygen species, the dye is converted to the fluorescent form of 2′,7′-dichlorofluorescein (DCF). A fluorescence microplate reader (SpectraMax M5, Molecular Devices Corporation, California, USA) was used to detect the generation of fluorescent DCF molecules with an excitation filter of 485 nm and an emission filter of 525 nm, with a 515 nm cutoff filter. Cells were plated in a 96-well black walled clear bottom plate at a density of 1-2×10⁴ cells per well, depending on the cell line. The cells were incubated at 37° C., 5% CO₂ overnight. Respective groups of cells were pretreated with C₆₀-ser (100 μM), tert-butyl hydroperoxide (TBHP; 200 μM) as a positive control, or N-acetyl cysteine (50 μM). N-acetyl cysteine (NAC) was a precursor to glutathione, a known antioxidant that serves in this example as a positive control. A 10 μM solution of H₂-DCFDA was prepared in phenol red free DMEM and applied to each well 30 minutes before radiation. The plates were wrapped in aluminum foil for transport to the radiation treatment center and maintained in the dark throughout radiation. Following irradiation, the cells were transported back for plate reading, 30 minutes post radiation.

### Results of Evaluation of C₆₀-Ser in Biological Systems Under Proton Irradiation

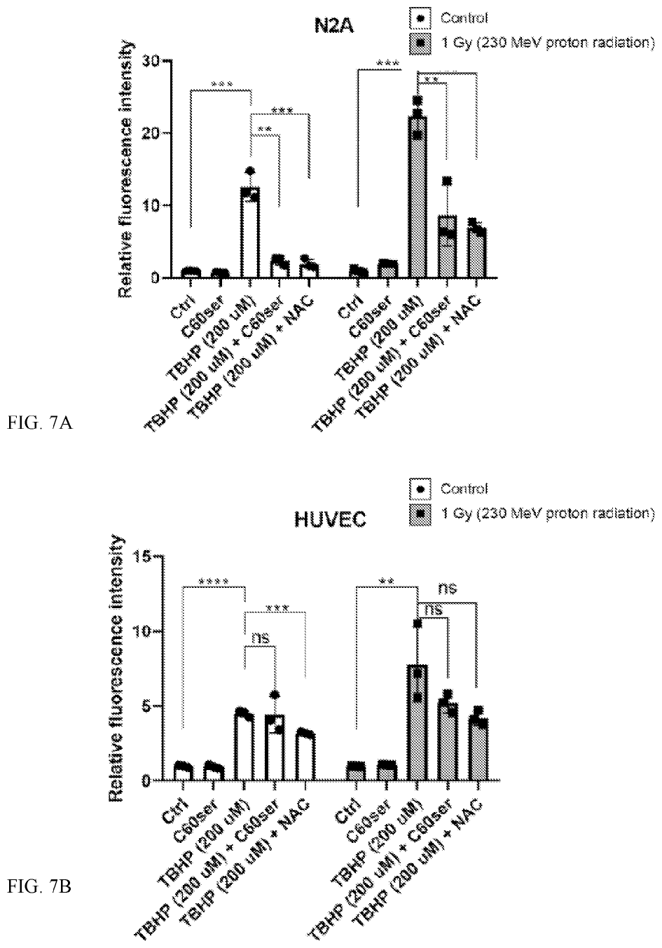

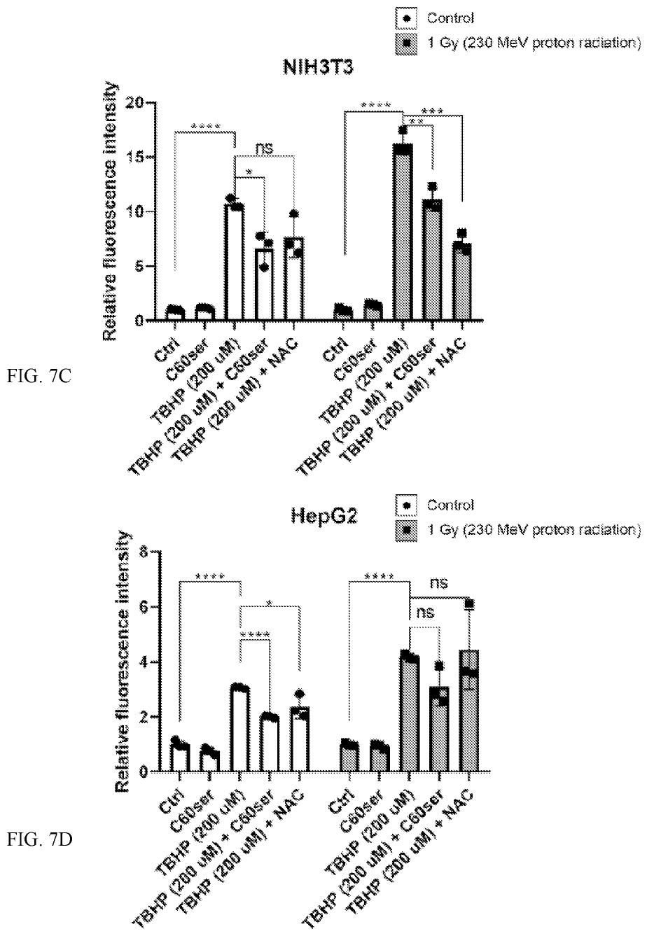

Free radical reactions that occur due to radiation occur in a matter of milliseconds. Accordingly, the amount of ROS generated from 1 Gy of proton radiation was not sustainable for long enough to see a significant increase in ROS generated when the cells were analyzed approximately 30 minutes post radiation (see, e.g., FIGS. 7A-7D). Radiation causes radiolysis of water, leading to the generation of multiple reactive oxygen species, for example, hydroxyl ions, hydroxyl radicals, and hydrogen peroxide. All of these reactive oxygen species are capable of oxidizing H₂-DCFDA. However, the cells not only intrinsically quenched ROS, but were also able to transport the fluorescein dye out of the cell, thus weakening the signal as time progresses. For three of the four cell lines that received the positive control TBHP, there was a significant decrease in the amount of ROS detected when cells were pretreated with C₆₀-ser compared to control, indicating that the C₆₀-ser ability to quench ROS and reduce ROS-mediated damage to cells.

Although there was no significant increase in ROS generated in cells irradiated with 1 Gy of proton radiation compared to control, there was a significant increase in ROS generated when positive controls were included. In other words, there was a significant increase in ROS generated in the irradiated samples treated with TBHP compared to the non-irradiated samples treated with TBHP. Without wishing to be bound by any particular theory, the amount of ROS generated due to irradiation of the cells treated with TBHP was likely too high for the cells to naturally quench in a 30-minute time window. Accordingly, the increase ROS due to radiation was detected in this group, as shown by the increased signal in the irradiated samples with TBHP compared to the non-irradiated samples treated with TBHP. The N2A (FIG. 7A) and NIH3T3 (FIG. 7C) cell lines show a significant reduction in ROS in the presence of C₆₀-ser. The positive control, NAC, also exhibited a decrease in ROS for N2A, HUVEC, and NIH3T3, as well as for the control HepG2 cells.

### Example 8—Assessment of DNA Damage

### Methods

To test the ability of C₆₀-ser to protect against radiation injury, the level of DNA damage in response to radiation was analyzed by measuring γH2AX. The H₂AX protein becomes phosphorylated into γH₂AX as a result of DNA double strand breaks. This allows for the visualization and quantification of double strand break foci through immunofluorescence analysis. Cells were plated on 8 chamber slides and incubated overnight before C₆₀-ser treatment. Three sets of slides per cell line (to be irradiated with 0, 0.5, and 1 Gy) were prepared, with the top 4 rows used as a control and the bottom 4 rows incubated with 100 μM C₆₀-ser. Cells were incubated with C₆₀-ser for 2 hours before irradiation with a clinical Proteus One proton machine (Ion Beam Applications, Louvain-la-Neuve, Belgium). A 230 MeV mono-energetic unmodulated broad beam of large field size that encompassed all slides containing the cells was used to deliver the 0.5 and 1 Gy doses. White solid water slabs (IBA Dosimetry, Germany) provided the necessary phantom material to place the cells at the desired 8 cm depth with an LET of approximately 1.7 keV/μm. After irradiation, cells were incubated at 37° C. with 5% CO₂ for 30 minutes before washing and fixing the cells with 1% formaldehyde. The cells were permeabilized with 1% NP-40 and incubated with 5% BSA to block unspecific antibody bonding. The cells were then incubated with γH₂AX primary antibody followed by a secondary antibody. Mounting media containing DAPI was used before applying cover slips and sealing the samples. Cells were imaged on an Olympus IX71 trinocular inverted fluorescence phase contrast microscope with a 40× objective. The resulting images were analyzed by ImageJ, and the foci were counted.

### Results of Assessment of DNA Damage

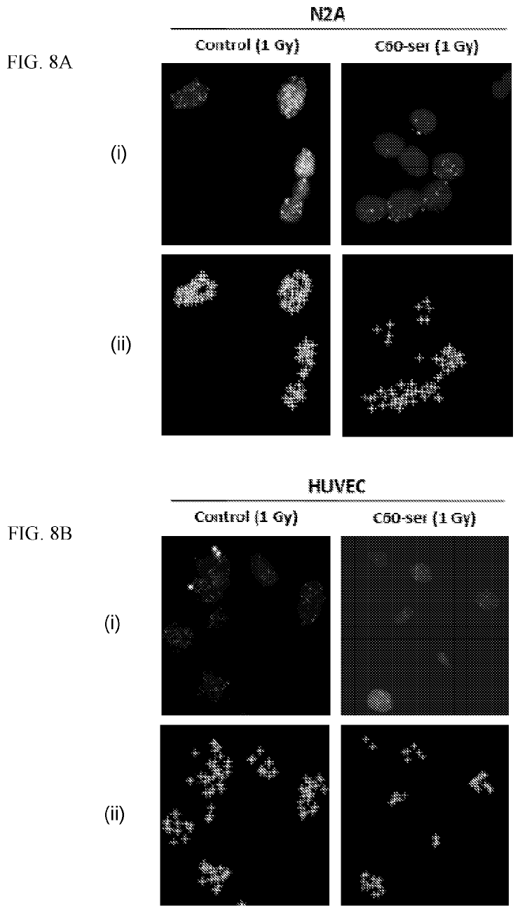

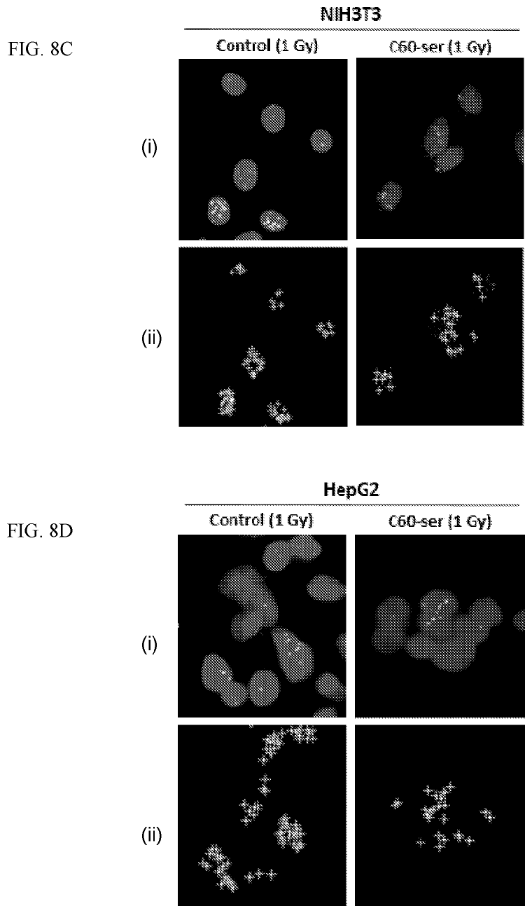

FIGS. 8A-D show example images of control and C₆₀-ser pretreated cells, with immunofluorescence staining of γH₂AX and DAPI images overlaid with each other (i) and γH₂AX foci alone (ii). ImageJ was used to threshold and count the number of foci. In the absence of radiation, there was no significant different in foci expression in C₆₀-ser treated groups compared to control. In groups treated with 0.5 Gy of radiation, there was a trend towards reduction in total foci formed, but it was not statistically significant across all four cell lines at the analyzed time point, as determined by Student's t-test. However, when cells were treated with 1 Gy of proton radiation, there was a significant decrease in the number of foci across all four cell lines, indicating a reduction in DNA damage when cells are pretreated with C₆₀-ser.

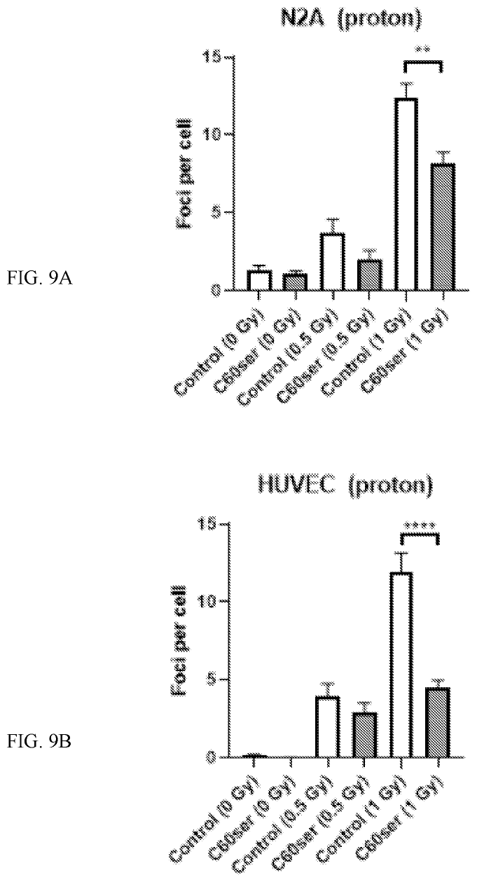

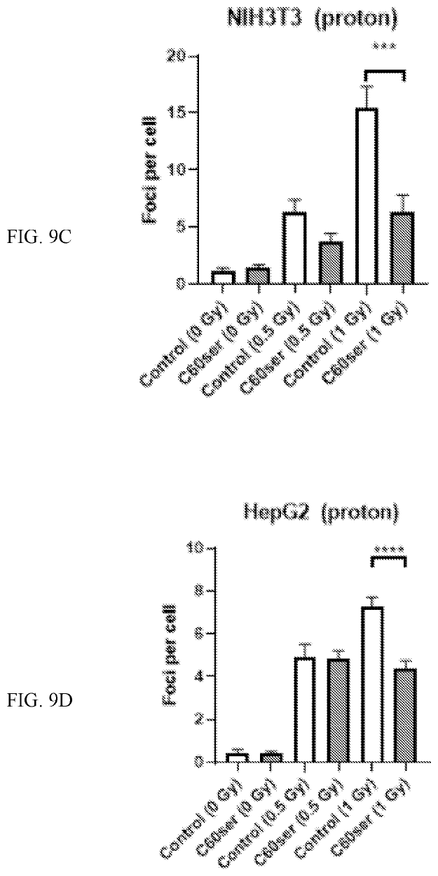

FIGS. 9A-D show the foci distribution of N2A (FIG. 9A), HUVEC (FIG. 9B), NIH3T3 (FIG. 9C), and HepG2 (FIG. 9D) cells following exposure to 0.5 and 1 Gy of 227 MeV proton radiation with or without 100 μM C₆₀-ser, where **=p<0.005, ***=p<0.0005, and ****=p<0.00005 (n=50 t 5 cells).

### Example 9—Examining Clonogenicity of Irradiated Cells

A classic clonogenics assay was performed to further test the ability of C₆₀-ser to protect against radiation damage. After irradiation, a fraction of the cells will undergo radiation-induced death, whereas other cells remain metabolically active and are able to undergo a few cycles of mitosis before meeting their endpoint. Since many cells remain metabolically activity, classic toxicity measurements such as an MTT measurement are not able to catch the full effects of radiation damage. In contrast, a classic clonogenics assay, which plates a known number of cells and tracks development of colonies, is able to track the number of cells that maintain their clonogenicity. With increasing radiation doses, the fraction of cells that maintain clonogenicity decrease. Therefore, a successful radioprotectant is able to increase the number of surviving colonies compared to the control at the same radiation doses.

### Methods

N2A, NIH3T3, and HepG2 cells were plated in 35 mm dishes and treated with 100 μM C₆₀-ser for 2 hours prior to washing with PBS and irradiation at 0, 2, 4, and 6 Gy with a 230 MeV proton beam. Following radiation, a known number of cells were re-plated in six well plates and allowed to grow in culture for 8-14 days to allow for colony formation. To account for cell death with higher radiation doses, a larger number of cells were plated with increasing doses received. Each cell line was optimized for the number of cells plated and the duration of growth post plating, to allow for successful colony formation. The colonies were fixed and stained with 1% crystal violet in ethanol. Next, the plates were washed and allowed to dry before manual counting of colonies. The survival curves were plotted after normalizing to the group without radiation, and the data was fitted to a linear-quadratic model.

### Results of Examining Clonogenicity of Irradiated Cells

FIGS. 10A-10C show the results of the classic clonogenics survival analysis of N2A (FIG. 10A), NIH3T3 (FIG. 10B), and HepG2 (FIG. 10C) cells pre-treated with C₆₀-ser. In all three cell lines, treatment with C₆₀-ser increased the fraction of surviving cells. The dose enhancement factors (DEFs) calculated at 20% surviving fraction were 0.91 (N2A, one set of clonogenics), 0.85±0.03 (NIH3T3), and 0.88±0.01 (HepG2).

### Example 10—Assessing Mitochondrial Respiration by Seahorse

### Methods

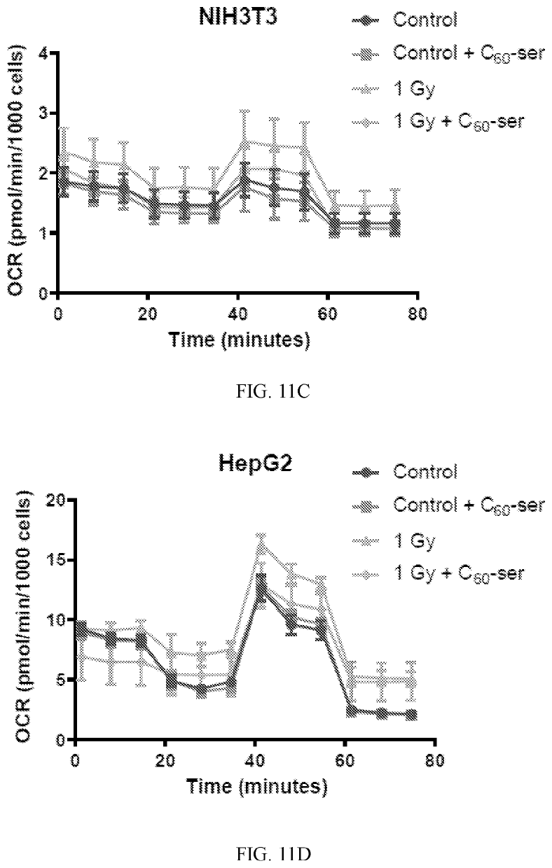

The oxygen consumption rates were assessed in N2A, HUVEC, NIH3T3, and HepG2 cell lines with a Seahorse XFe96 (Agilent Technologies, Santa Clara, CA, USA). Cells were treated with 0 and 1 Gy of 230 MeV proton irradiation. The control plates were run at about the same time post plating. Each cell line was optimized prior to these experiments, and data was presented normalized to cell number plated. The standard mitochondrial stress test was performed using serial injections of oligomycin (1.5 μM), carbonyl cyanide p-(trifluoromethoxy)phenylhydrazone (FCCP, 1 μM), and rotenone/antimycin A mixture (0.5 μM). After injection of oligomycin, which inhibits ATP synthase (complex V), there was a drop in electron flow through the electron transport chain (ETC) and a resultant drop in oxygen consumption rate (OCR) of the cells. Injection of FCCP collapsed the proton gradient and allowed for uninhibited electron flow through the ETC supercomplexes. At this point in the experiment, the complex IV of the ETC reached its maximum, which can be noted by the increase in the OCR, as shown in FIGS. 11A-D. The injection of inhibitors of complex I (rotenone) and complex III (antimycin A) shut down the mitochondrial respiration and allowed for measurement of non-mitochondrial respiration. The reserve respiratory capacity was calculated by subtracting the basal respiratory capacity from the maximal respiratory capacity.

### Results for Assessing Mitochondrial Respiration by Seahorse

The basal oxygen consumption rate was measured approximately 30 minutes post irradiation, in which case there was either a slight increase or no change in oxygen consumption rate in the irradiated groups compared to the control groups. However, across all four cell lines, there was an increase in maximum respiratory capacity and reserve capacity in groups irradiated with 1 Gy of 230 MeV proton radiation compared to un-irradiated control. Without wishing to be bound by any particular theory, ionizing radiation can induce mitochondrial elongation and increase electron transport chain supercomplexes, which translates to increased reserved respiratory capacity. It is likely that the increase in maximum respiratory capacity in response to radiation was the result of cells that were preparing to meet the increased energy demand due to the damage caused by the irradiation. This increase in respiratory capacity was dampened and, in some cases, reduced to normal or slightly below normal levels when cells were pretreated with C₆₀-ser for 2 hours before irradiation. These results further illustrate that C₆₀-ser serves as a radioprotectant, as reduced damage results in less energy demand and less mitochondrial reprogramming.

### Example 11—Whole Body Radiation Protection

### Methods

C57B16 mice were pretreated with a per os gavage of C₆₀-ser before exposure to radiation. Mice were treated with either once with 700 mg/kg 12 hours before exposure, or 700 mg/kg C₆₀-ser 12 hours before exposure, followed by 70 mg/kg treatments daily for eight days. Mice were given whole-body radiation with an x-ray irradiator at a dose of 9.5 Gy.

### Results of Whole Body Radiation Protection

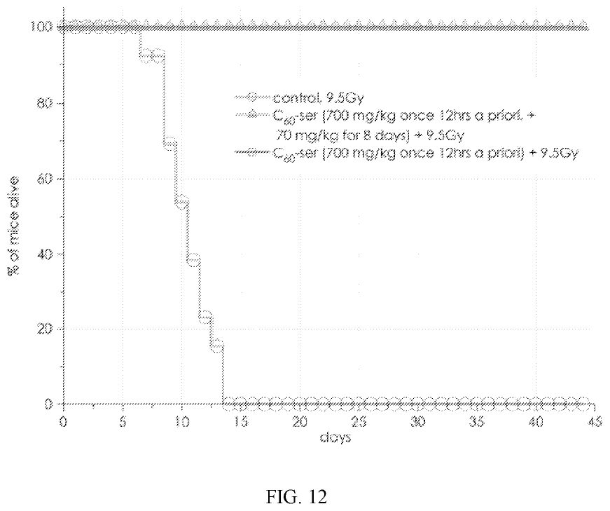

FIG. 12 shows the percentage of mice in the control and treatment groups as a function of time. Both treatment groups experienced 100% survival over the course of a 45 day study. In contrast, control mice did not survive past day 14 of the study. Accordingly, a single dose of C₆₀-ser prevented radiation induced death, further illustrating the protective abilities of C₆₀-ser.

### Example 12—In Vitro Effects on Tumor Cells

### Methods

A clonogenic assay of HCT116 colorectal cancer cells was conducted. 2×10⁶ cells were treated with 100 μM C₆₀-ser for 2 hours and exposed to 0-6 Gy of radiation.

Results of In vitro Effects on Tumor Cells

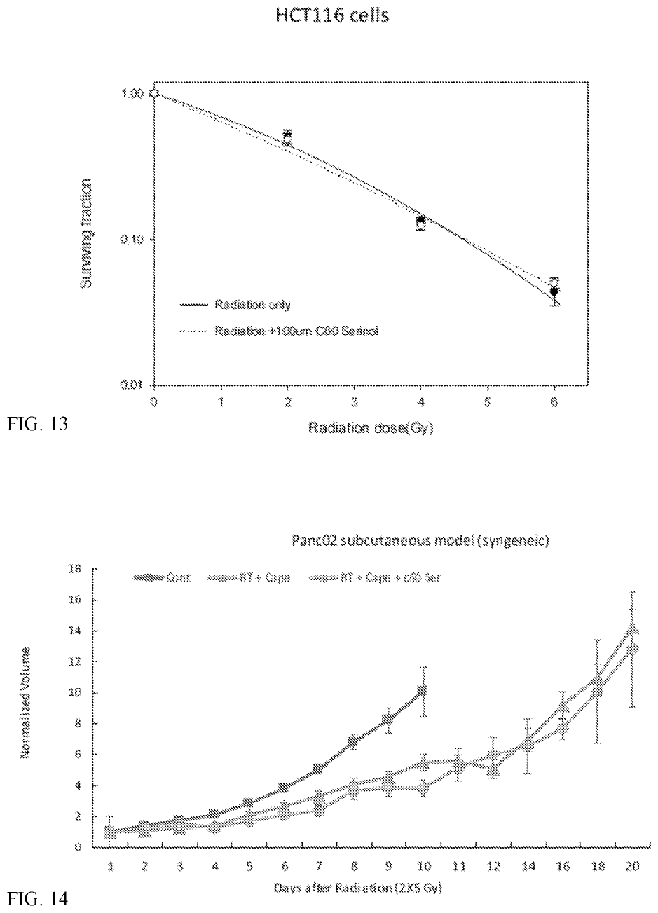

FIG. 13 shows the result of the HCT116 clonogenics assay. There was no difference in the surviving fraction of cells in the control cells relative to the cells treated with C₆₀-ser, indicating that C₆₀-ser does not appear to protect these tumor cells from radiation induced cell death.

### Example 13—In Vivo Effects on Tumor Cells

### Methods

A syngeneic mouse model of pancreatic cancer (Panc02 growing on the thigh of C57B16 mice) was used to evaluate the effects of C₆₀-ser on in vivo tumors. Mice were treated with 350 mg/kg capecitabine via per os gavage for 5 days 1 hour before radiation (RT+Cape), or capecitabine treatment with a single dose of 700 mg/kg C₆₀-ser via per os gavage once 12 hours before the first dose of radiation (RT+Cape+c60 Ser). Mice were exposed to 2 Gy of radiation (RT) once per day for 5 days. A control group (Cont) without treatment also was exposure to the radiation treatment. Normalized tumor volumes were monitored for 20 days.

### Results of Analysis of In Vivo Effects on Tumor Cells

As shown in FIG. 14, mice treated with capecitabine and C₆₀-ser were not significantly different in normalized tumor volume, compared to mice treated with capecitabine alone. Therefore, C₆₀-ser does not appear to offer radioprotection to the Panc02 tumor cells.

### Terms

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “solvent” as used herein refers to a liquid that can dissolve a solid, another liquid, or a gas to form a solution. Non-limiting examples of solvents are silicones, organic compounds, water, alcohols, ionic liquids, and supercritical fluids.

As used herein, “weight percent” (wt %) can be considered a mass fraction or a mass ratio of a substance to the total mixture or composition. Weight percent can be a weight-to-weight ratio or mass-to-mass ratio, unless indicated otherwise.

The following units of measure have been mentioned in this disclosure:

| Unit of Measure | Full form |
|---|---|
| ° C. | degrees Celsius |
| cm | centimeter |
| μm | micrometer |
| nm | nanometer |
| MeV | mega electron volt |
| keV | kiloelectron volt |
| μS/cm | microsiemens per centimeter |
| Gy | Gray |
| μM | micromolar |
| mg | milligram |
| kg | kilogram |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for prolonging survival of a mammal exposed to a lethal level of radiation, wherein said method comprises administering a composition comprising C₆₀-serinol to said mammal, wherein said mammal survives longer than if said mammal was not administered said composition.

2. The method of claim 1, wherein said mammal is human.

3. The method of claim 1, wherein said lethal level of radiation is greater than 5 Gy.

4-8. (canceled)

9. A method for prolonging survival of a mammal expected to be exposed to a lethal level of radiation, wherein said method comprises administering a composition comprising C₆₀-serinol to said mammal before said mammal is exposed to said lethal level of radiation, wherein said mammal survives longer than if said mammal was not administered said composition.

10. The method of claim 9, wherein said mammal is human.

11. The method of claim 9, wherein said lethal level of radiation is greater than 5 Gy.

12-16. (canceled)

17. A method for reducing the severity of radiation-induced injury, wherein said method comprises administering a composition comprising C₆₀-serinol to a mammal exposed to a damaging level of radiation, wherein the severity of radiation-induced injury of said mammal is less than the severity of radiation-induced injury if said mammal was not administered said composition.

18. The method of claim 17, wherein said mammal is human.

19. The method of claim 17, wherein said damaging level of radiation is greater than 0.5 Gy.

20-24. (canceled)

25. A method for reducing the severity of radiation-induced injury, wherein said method comprises administering a composition comprising C₆₀-serinol to a mammal prior to exposure to a damaging level of radiation, wherein said mammal is exposed to said damaging level of radiation, and wherein the severity of radiation-induced injury of said mammal is less than the severity of radiation-induced injury if said mammal was not administered said composition.

26. The method of claim 25, wherein said mammal is human.

27. The method of claim 25, wherein said damaging level of radiation is greater than 0.5 Gy.

28-32. (canceled)

33. A method for treating a mammal to reduce the number of reactive oxygen species in said mammal following radiation exposure, wherein said method comprises administering a composition comprising C60-serinol to said mammal before, during, or after exposure to a damaging level of radiation, wherein said mammal is exposed to said damaging level of radiation, and wherein the number of reactive oxygen species in said mammal following said radiation exposure is less than the number of reactive oxygen species in said mammal if said mammal was not administered said composition.

34. The method of claim 33, wherein said mammal is human.

35. The method of claim 33, wherein said damaging level of radiation is greater than 0.5 Gy.

36-43. (canceled)

44. A composition comprising C₆₀-serinol.

45. The composition of claim 44, wherein said composition further comprises an aqueous or non-aqueous sterile injection solution.

46. (canceled)

47. A method of making C₆₀-serinol, wherein said method comprises:
(a) reacting serinol malonate tetraacetate with [60]fullerene in the presence of 1,8-diazabicyclo[5.4.0]undec-7-ene and perbromomethane in toluene to yield C₆₀-serinol acetate; and
(b) reacting said C₆₀-serinol acetate with hydrochloric acid in an aqueous solution of dioxane to yield C₆₀-serinol.

48. The method of claim 47, wherein reacting serinol malonate tetraacetate with [60]fullerene comprises reacting serinol malonate tetraacetate and [60]fullerene in a 6:1 to 15:1 molar ratio.

49. (canceled)

50. The method of claim 47, wherein said method comprises reacting serinol malonate with acetic anhydride to yield serinol malonate tetraacetate.

51. (canceled)
